(19) United States
(12) Patent Application Publication
(10) Pub. No.: US 2026/0096934 A1
(43) Pub. Date: Apr. 9, 2026

(54) COMFORT SLEEVE FOR MALE INCONTINENCE

(71) Applicant: Eric Lee, Laguna Niguel, CA (US)

(72) Inventor: Eric Lee, Laguna Niguel, CA (US)

(21) Appl. No.: 19/121,735

(22) PCT Filed: Jun. 15, 2023
(86) PCT No.: PCT/US2023/025426

§ 371 Date: Apr. 16, 2025

Related U.S. Application Data
(60) Provisional application No. 63/417,575, filed on Oct. 19, 2022.

(51) Int. Cl. A61F 13/471; A61F 13/15; A61F 13/53; A61F 13/539; A61F 13/64

(52) U.S. Cl. CPC A61F 13/471; A61F 13/15203; A61F 13/539; A61F 13/64; A61F 2013/15146; A61F 2013/15243; A61F 2013/530481; A61F 2013/53908

(57) ABSTRACT

A comfort sleeve for male incontinence has a sleeve with an outer layer and an inner layer with an absorbent disposed between them. The sleeve has an upper compression zone disposed along a top portion; a lower compression zone disposed along a bottom portion of the sleeve and a contour compression zone disposed between the upper compression zone and the bottom compression zone. The upper compression zone has at least one elastic band with a first elasticity; the contour compression zone has at least one elastic band with a second elasticity where the second elasticity is selected to comfortably compress the sleeve around the base of the glans of a penis; thereby removably securing the sleeve around said penis. The bottom compression zone is selected to have a third elasticity wherein each selected elasticity provides a compressive force selected to comfortably shape and hold the sleeve around the penis.

### CROSS REFERENCE TO RELATED APPLICATIONS

This application claims priority and herein incorporates by reference U.S. provisional patent application 63417575, filed 19 Oct. 2022.

Male incontinence is a very common problem with estimates ranging from 25 to 30 percent of United States males suffer from it. In addition to the medical condition, incontinence can also have emotional, psychological and social implications as well. Sufferers of male incontinence can be afraid to stray too far from a restroom or shy away from being socially active due to the possible embarrassment of having an incident.

Many treatments to the problem of male incontinence are available including catheters, external collecting systems, drainage bags, and absorbent products such as absorbent underwear. Many people are hesitant to utilize these treatments since they can be inconvenient and may cause additional embarrassment when the user uses a public restroom facility.

There is a need for a simple, comfortable device that treats male incontinence while being discrete and aesthetically attractive.

### BACKGROUND OF THE INVENTION

Male incontinence is a very common problem with estimates ranging from 25 to 30 percent of United States males suffer from it. In addition to the medical condition, incontinence can also have emotional, psychological and social implications as well. Sufferers of male incontinence can be afraid to stray too far from a restroom or shy away from being socially active due to the possible embarrassment of having an incident.

Many treatments to the problem of male incontinence are available including catheters, external collecting systems, drainage bags, and absorbent products such as absorbent underwear. Many people are hesitant to utilize these treatments since they can be inconvenient and may cause additional embarrassment when the user uses a public restroom facility.

There is a need for a simple, comfortable device that treats male incontinence while being discrete and aesthetically attractive.

### SUMMARY OF THE INVENTION

A comfort sleeve for male incontinence has a sleeve with an outer layer and an inner layer with an absorbent disposed between them. The sleeve has an upper compression zone disposed along a top portion; a lower compression zone disposed along a bottom portion of the sleeve and a contour compression zone disposed between the upper compression zone and the bottom compression zone. The upper compression zone has at least one elastic band with a first elasticity; the contour compression zone has at least one elastic band with a second elasticity where the second elasticity is selected to comfortably compress the sleeve around the base of the glans of a penis; thereby removably securing the sleeve around said penis. The bottom compression zone is selected to have a third elasticity wherein each selected elasticity provides a compressive force selected to comfortably shape and hold the sleeve around the penis.

Other features and advantages of the instant invention will become apparent from the following description of the invention which refers to the accompanying drawings.

### BRIEF DESCRIPTION OF THE DRAWINGS

FIG. 1 is an illustration of a Comfort sleeve for male incontinence according to an embodiment of the invention.

FIG. 2 is a cutaway view of the comfort sleeve shown in FIG. 1.

FIG. 3 is an illustration of the comfort sleeve shown in FIG. 1 in use.

FIG. 4 is a cutaway view showing the layers of the comfort sleeve shown in FIG. 1.

FIG. 5 is an illustration of the comfort sleeve shown in FIG. 1 in a manufacturing configuration showing the layer composition.

FIG. 6 is an illustration of the comfort sleeve shown in FIG. 6 with a plurality of elastic portions applied thereon.

FIG. 7 is an illustration of the comfort sleeve show in FIG. 6 being folded along a center portion.

FIG. 8 is an illustration of the comfort sleeve shown in FIG. 7 being sealed and cut to produce the final product.

FIG. 9 is a side view showing the layers of the comfort sleeve shown in FIG. 5.

### DETAILED DESCRIPTION OF THE INVENTION

In the following detailed description of the invention, reference is made to the drawings in which reference numerals refer to like elements, and which are intended to show by way of illustration specific embodiments in which the invention may be practiced. It is understood that other embodiments may be utilized and that structural changes may be made without departing from the scope and spirit of the invention.

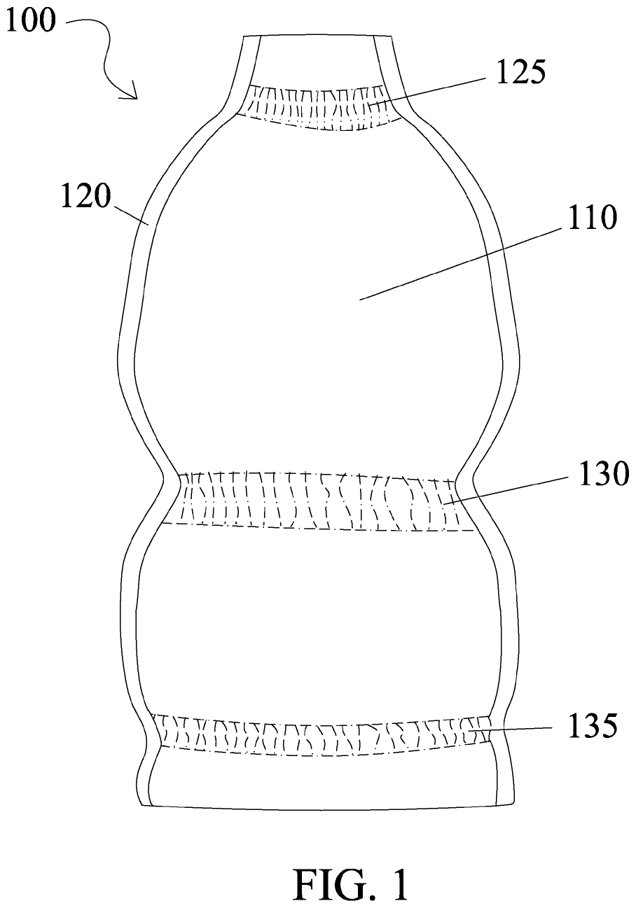

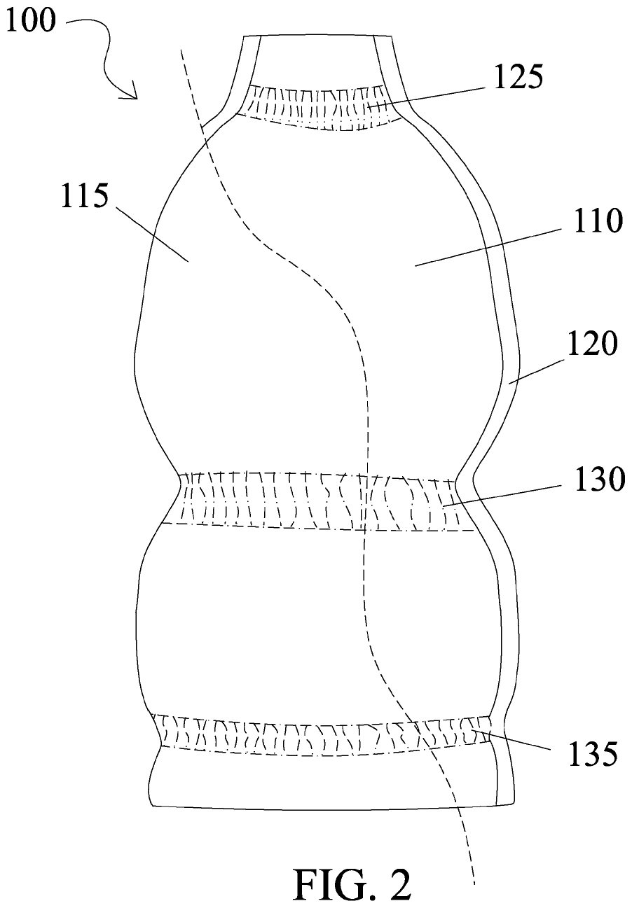

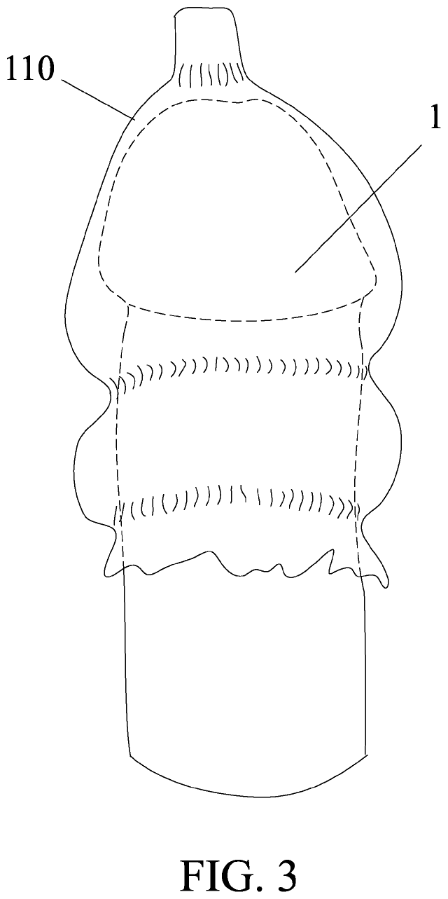

Referring to FIGS. 1-3, a comfort sleeve for male incontinence 100 is shown having an outer layer 110, an inner layer 115, a sealed edge 120 and a lower compression zone 135, a contour compression zone 130 and an upper compression zone 125. Outer sleeve 110 is a laminate made of a 14 gsm (grams per square meter) Poly and a 12 gsm spunbond fabric providing a strong yet soft sheath that is comfortable to wear. Of course other fabrics may be used as long as a strong yet comfortable material is chosen. In order to provide a comfortable yet liquid containing sleeve, the compression zones are selected to provide a different compressive value by adjusting the number of strands used as well as the weight of the elastic used. To provide upper compression zone 125 compressive force to seal off a top portion of comfort sleeve 100, two strands of 1100 decitex spandex are used with a 300% elongation. Alternatively, the top of Sleeve 100 could be sealed across the top (not shown) eliminating compression zone 125. Contour compression zone 130 typically uses two strands of 900 decitex Spandex at 200 % elongation. Lower compression zone 135 is wider than the other two zones and uses five strands of 500 decitex Spandex to further enhance comfort. Of course other materials, percentages of elongation and number of strands may be used as long as comfort sleeve 100 fits comfortably yet secure in use.

Inner layer 115 is provided to comfortably fit over a penis 140. Inner layer 115 is a 12-14 gsm spunbond material which provides the requisite softness and comfort allowing it to be placed in direct contact with the user's skin. Outer layer 110 may be tinted to appear more natural in color to the user's natural skin tone to aid in the aesthetic appearance which may reduce the user's embarrassment or other feeling while wearing and using an incontinence device.

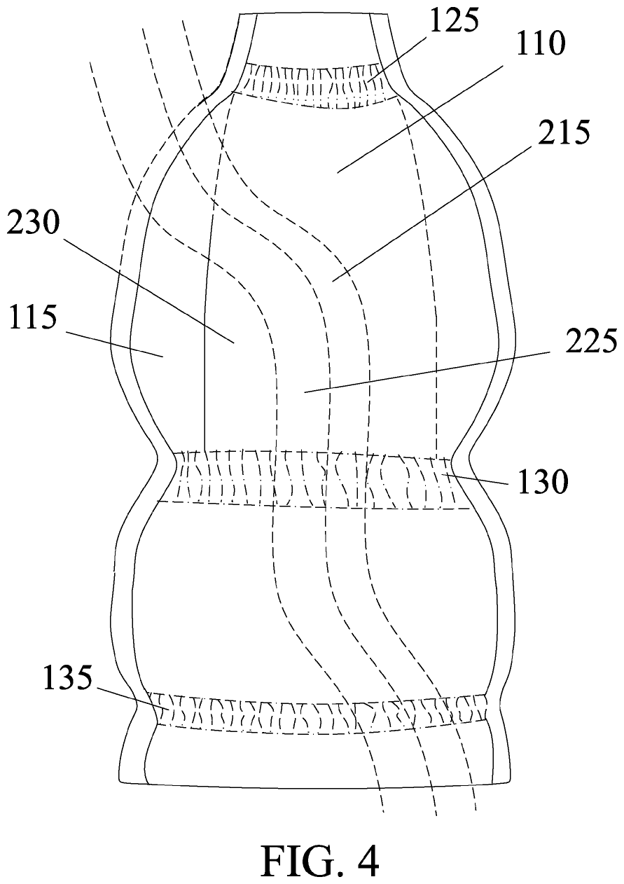

Referring to FIG. 4, a cutaway view of comfort sleeve 100 is shown having outer layer 110 with an adhesive layer 215, upper compression zone 125, contour compression zone 130, lower compression zone 135, a melt spray adhesive layer 225, an absorbent layer 230 and then inner layer 115. Absorbent layer 230 is either Gelok or Airlaid NW (Non-woven) superabsorbent polymer (SAP). Outer layer 110 is a hydrophobic polyethylene barrier that may be breathable or non-breathable. Inner layer 115 is spunbond which is a cloth-like permeable material that provides a comfortable feel against the user's skin.

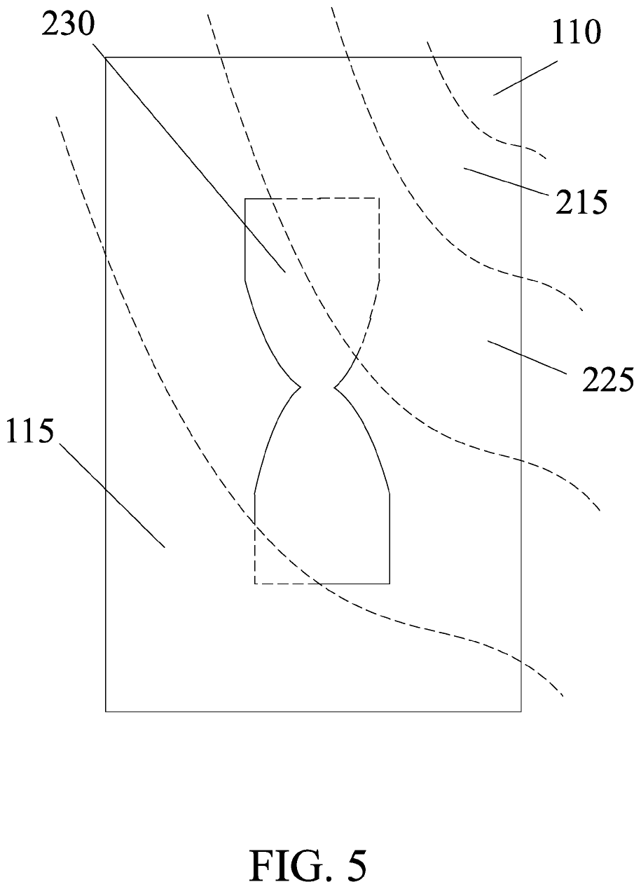

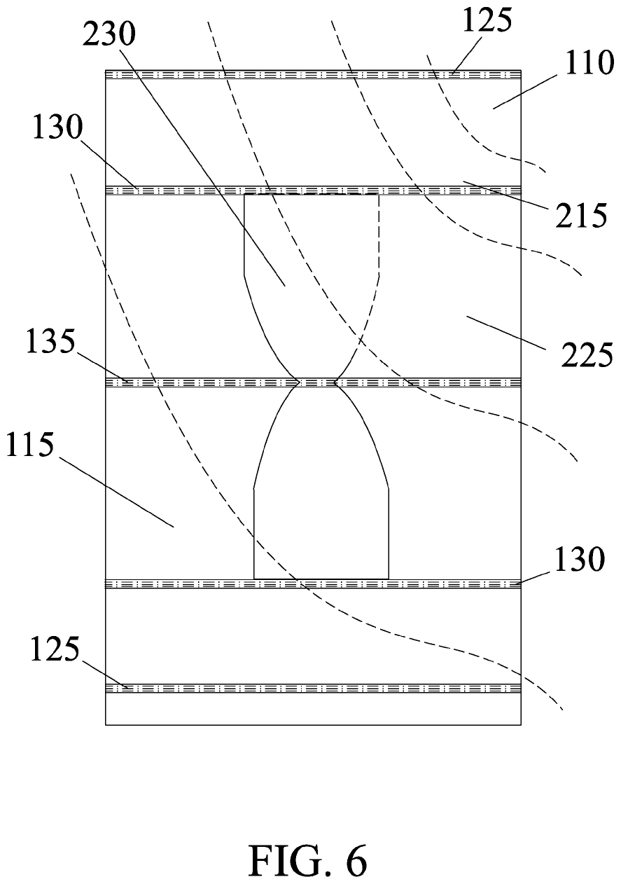

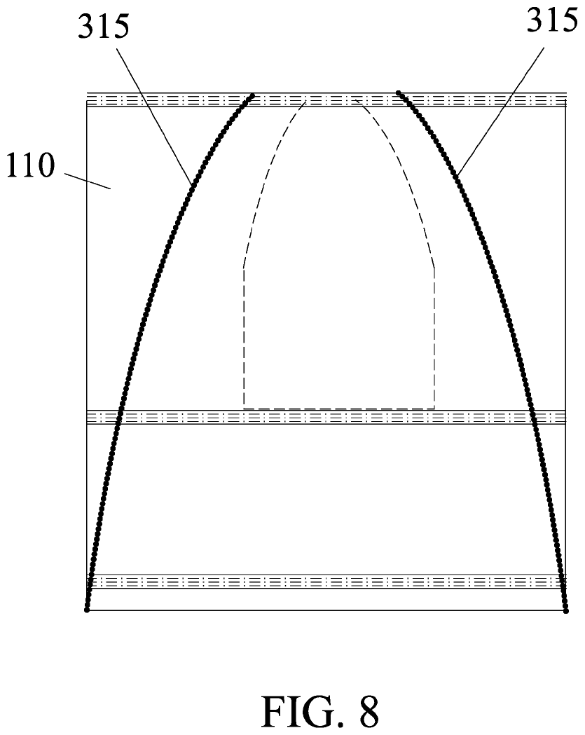

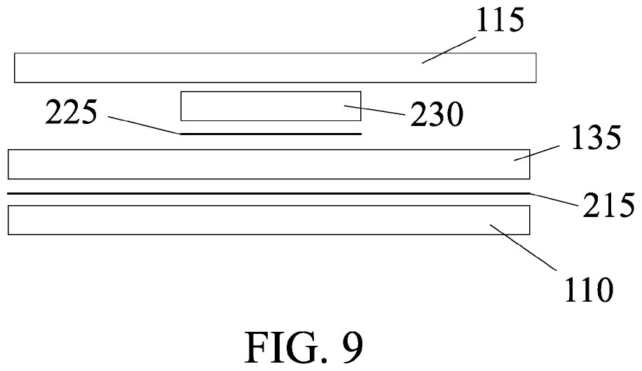

Referring now to FIGS. 5-9, comfort sleeve 100 is shown during the manufacturing process. To produce comfort sleeve 100, inner layer 115 starts as a rectangular sheet of 12-14 gsm spunbond. Absorbent layer 230 is a Gelock or Airlaid NW SAP material to capture and retain urine. As shown, absorbent layer 230 is precut to an hour-glass shape, however, it is possible to use other shapes as long as some absorbent material surrounds the tip of penis 140. compression zones, 125, 130 and 135 are secured with a melt spray pressure sensitive adhesive (PSA) under tension and outer layer 110 covers the other layers.

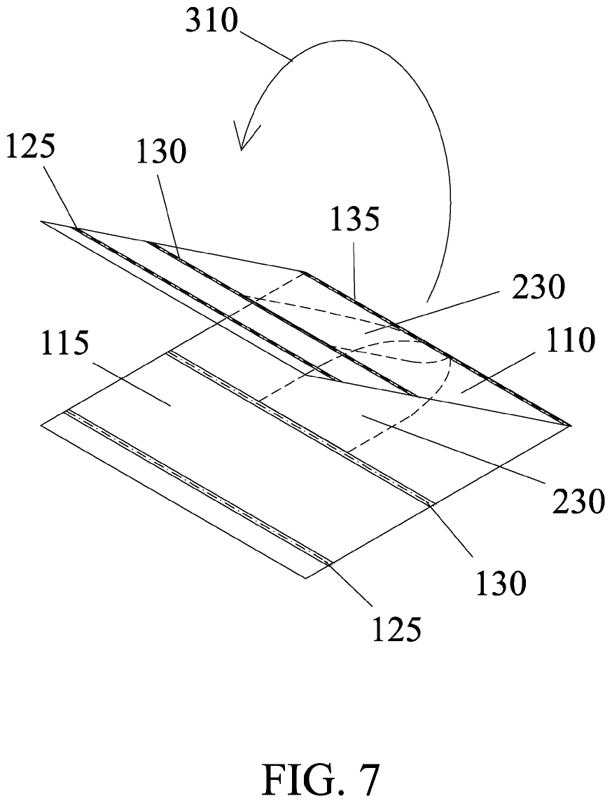

As shown in FIG. 7, the above construction is folded in half 310 with compression zones 125, 130 and 135 still under tension and then ultrasonically or thermally sealed and die cut 315 to produce the finished product. Once released, the tension from the zones retract to form the final shape. Because the compression zones are of different elastic properties, the upper portion of comfort sleeve greatly retracts the tip portion while the lower zone has more strands to form a comfortable “collar” portion that is comfortable. The contour zone's elasticity is selected to have a compression value between the upper zone and the lower zone to comfortably follow the contour of penis 140 and fits beneath the glans of penis 140.

It would be possible to add additional compression zones to further promote security if desired. compression zones are generally made of spandex as is known in the art; however other elastomeric materials may be used as long as the required compression levels are achieved. Additionally, it is possible to create two separate laminates as described above and layer them on top of each other to create the sleeve, thus eliminating the need to fold the product. Basically, this embodiment would look similar to the comfort sleeve shown in FIG. 7, except instead of folding one larger layered construction, two layered constructions would be placed together and then sealed and die cut as discussed above.

Although the instant invention has been described in relation to particular embodiments thereof, many other variations and modifications and other uses will become apparent to those skilled in the art.

What is claimed is:

1. A comfort sleeve for male incontinence comprising:
a sleeve;
said sleeve having an outer layer and an inner layer;
an absorbent disposed between said outer layer and said inner layer;
an upper compression zone disposed along a top portion of said sleeve;
a lower compression zone disposed along a bottom portion of said sleeve and a contour compression zone disposed between said upper compression zone and said bottom compression zone;
said upper compression zone having at least one elastic band selected to have a first elasticity;
said contour compression zone having at least one elastic band selected to have a second elasticity;
said second elasticity selected to comfortably compress said sleeve around the base of the glans of a penis; thereby removably securing said sleeve around said penis;
said bottom compression zone selected to have a third elasticity; and
wherein each selected elasticity provides a compressive force selected to comfortably shape and hold said sleeve around said penis.

2. The comfort sleeve according to claim 1 wherein said first elasticity comprises strands of 1100 decitex spandex having a 300% elongation from 1 to 5 strands.

3. The comfort sleeve according to claim 2 wherein said second elasticity comprises strands of 900 decitex spandex having a 200 % elongation from 1 to 5 strands.

4. The comfort sleeve according to claim 3 wherein said third elasticity comprises strands of 500 decitex spandex from 1 to 5 strands.

5. The comfort sleeve according to claim 1 wherein said outer is tinted with a selected color to match said user's skin color.

6. The comfort sleeve according to claim 1 wherein said absorbent is a super absorbent polymer.

7. The comfort sleeve according to claim 1 further comprising a first melt adhesive between said outer layer and said inner layer wherein said sleeve is bonded together.

8. The comfort sleeve according to claim 1 wherein said outer layer is a hydrophobic polyethylene barrier to contain liquid therein.

9. The comfort sleeve according to claim 1 wherein said absorbent is an absorbent laminate.
